(19) United States
(12) Patent Application Publication
(10) Pub. No.: US 2012/0259390 A1
(43) Pub. Date: Oct. 11, 2012

(54) Wearable Photvoltaic Ultra-Low frequency Acupoint Stimulator

(75) Inventor: Steven Clay Canion, Port Aransas, TX (US)

(73) Assignee: Steven Clay Canion, Port Aransas, TX (US)

(21) Appl. No.: 13/082,574

(22) Filed: Apr. 8, 2011

(51) Int. Cl. A61N 1/36

(52) U.S. Cl. 607/72

(57) ABSTRACT

The output of the oscillator is a series of electrical pulses that are typically in the range of 1 to 300 Hz. The open-circuit voltage across the electrodes is typically in the range of 1 to 60 volts, but is current-limited to values that are typically less than 500 uA. These levels are sufficient to provide a safe therapeutic response to the user. The embodiment shown here uses a Clare CPC1824 photovoltaic array and a Texas Instruments LPV7215 comparator configured in a standard oscillator circuit. Other embodiments could use other forms of photovoltaic array. The embodiment shown here is a flat, circular device with a diameter of less than 15 mm and a thickness of less than 5 mm. This makes it well suited as a wearable device that can remain in place for long periods while the user goes about their normal activities.

### TECHNICAL FIELD OF THE INVENTION

This invention relates to the field of electrical devices used to stimulate specific acupoints on the body of a human or animal. Such devices are commonly used as an alternative to traditional acupuncture needles. Electrical acupoint stimulators typically pass a small electrical current through a localized area on the skin to stimulate a selected acupoint.

### BACKGROUND

The field of acupuncture is well established and widely known. Traditional acupuncture typically involves the insertion of small needles at certain specific locations on the skin. These locations are referred to as acupoints. Various acupoints identified in the literature are considered to be associated with the treatment of specific mental and physical conditions. A sub-field of acupuncture, known as Auriculotherapy, was developed by the French neurologist Paul Nogier. Auriculotherapy specifically relates to stimulating acupoints on the human ear.

As an alternative to the use of traditional acupuncture needles, various methods have been developed for passing a small electrical current through an acupoint to stimulate the acupoint. Currently available products that do this include the Stim-Flex 400A from Electro Medical Inc., and the BioStim Plus from Lhasa Oms.

Currently available products tend to be physically large, are powered by either commercial AC power or by batteries, and are designed to be used by doctors. These characteristics limit their use to clinical settings such as doctor's offices and clinics. Because of these limitations, a typical therapy session using existing stimulators is limited to about 30 minutes.

There is some evidence that extending the time period that the stimulus is applied can have a beneficial effect, but currently available products are not well suited for extended use outside of a doctor's office or a clinic.

Thus, there exists a need for a small, wearable stimulator that can be used for extended periods without interfering with the patient's normal activities. Ideally, such a device would be simple enough for a user to operate and maintain on their own.

### SUMMARY OF THE INVENTION

The above-referenced deficiencies in the prior art are addressed by the present invention, which provides a small, wearable, acupoint stimulator that is powered by a photovoltaic array. The present invention is significantly smaller than currently available devices, and requires no external source of power.

### DETAILED DESCRIPTION OF INVENTION

A photovoltaic array (sometimes referred to as a solar cell) is attached to a small circuit board that contains an oscillator circuit and two electrically conductive electrodes. The photovoltaic array converts light into a source of electrical power for the oscillator circuit. The output of the oscillator circuit is applied across two conductive electrodes on the underside of the board.

When the two electrodes are placed in contact with the skin, a very small electrical current passes from one electrode, through the skin, to the other electrode. The unit can be placed on the skin and taped in place, or it can be held on an ear acupoint by placing a small magnet on the opposing side of the ear.

The output of the oscillator is a series of electrical pulses that are typically in the range of 1 to 300 Hz. The open-circuit voltage across the electrodes is typically in the range of 1 to 60 volts, but is current-limited to values that are typically less than 500 uA. These levels are sufficient to provide a therapeutic response, but are not high enough to present an electrical safety hazard to the user.

The figures show the construction of one embodiment of the invention.

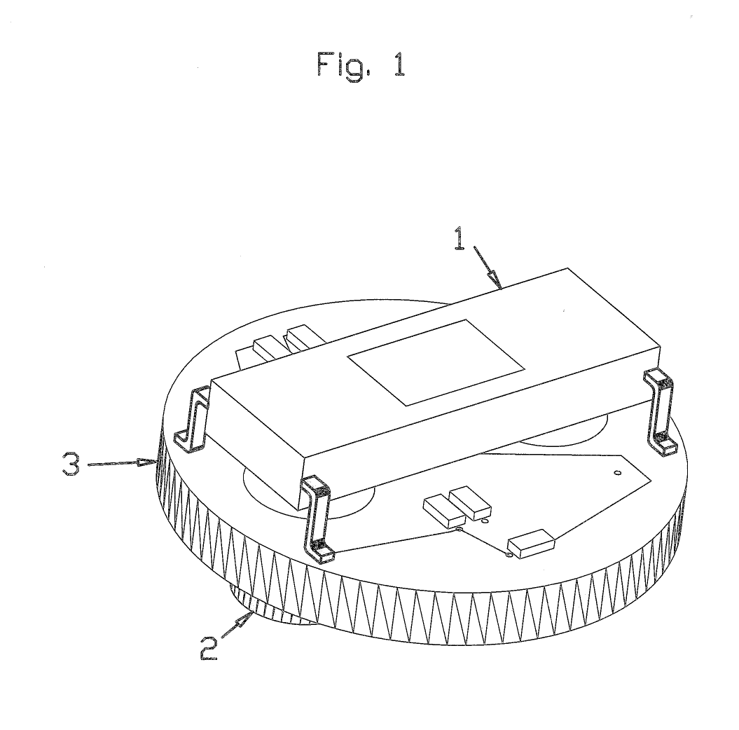

FIG. 1 shows: (1) photovoltaic array, (2) electrode, and (3) circuit board.

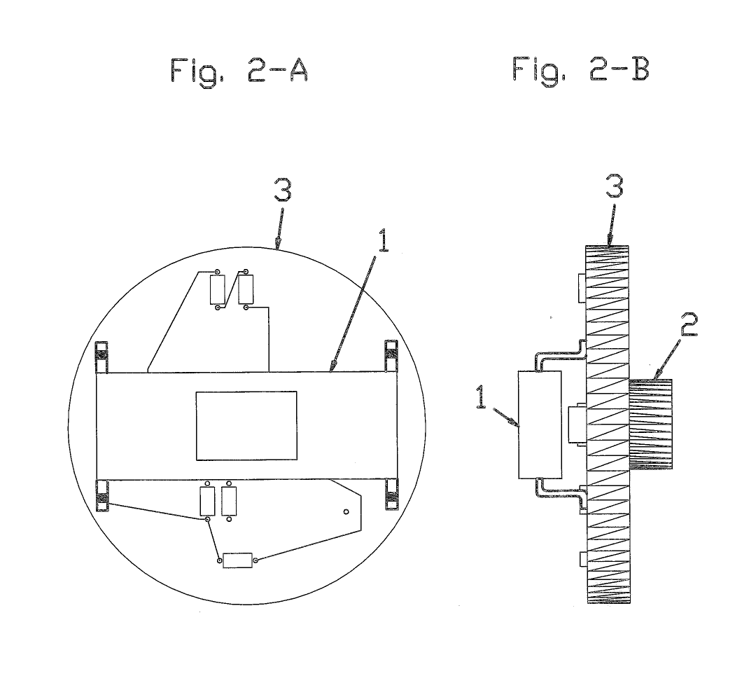

FIG. 2 shows: (1) photovoltaic array, (2) electrode, and (3) circuit board with oscillator circuit.

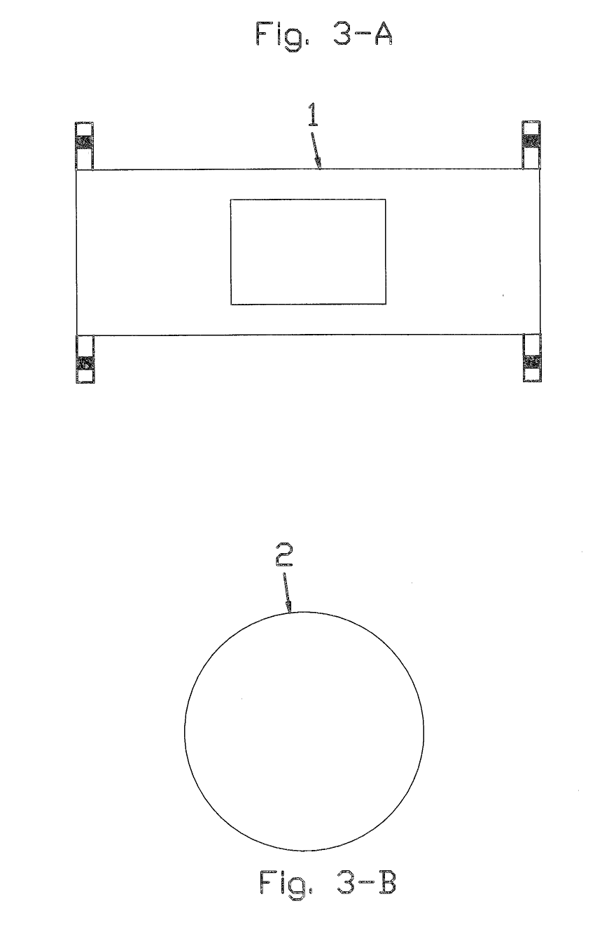

FIG. 3 shows the dimensions of the photovoltaic array (1) and the diameter of a representative electrode (2).

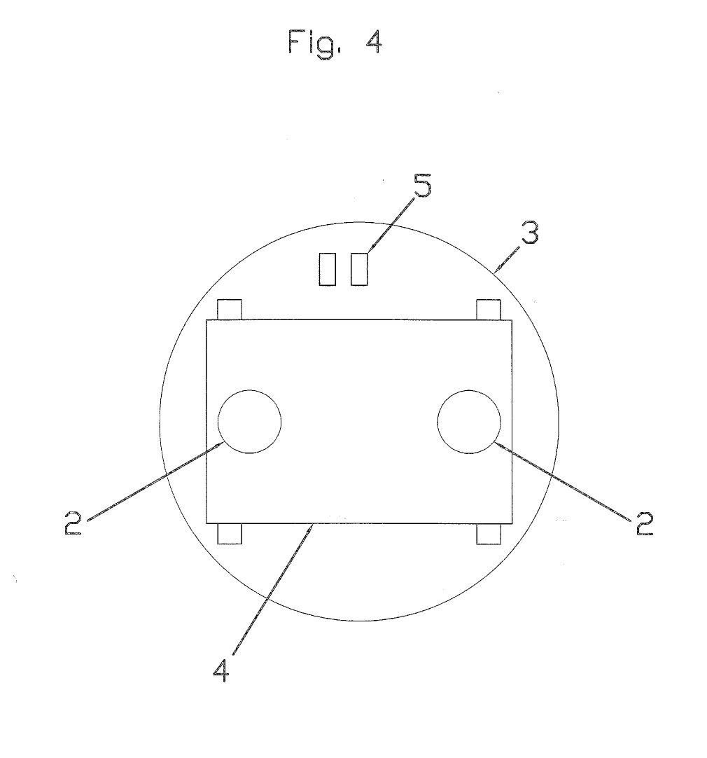

FIG. 4 shows the arrangement on the circuit board (3) of the two electrodes (2) and the oscillator circuit components (4, 5).

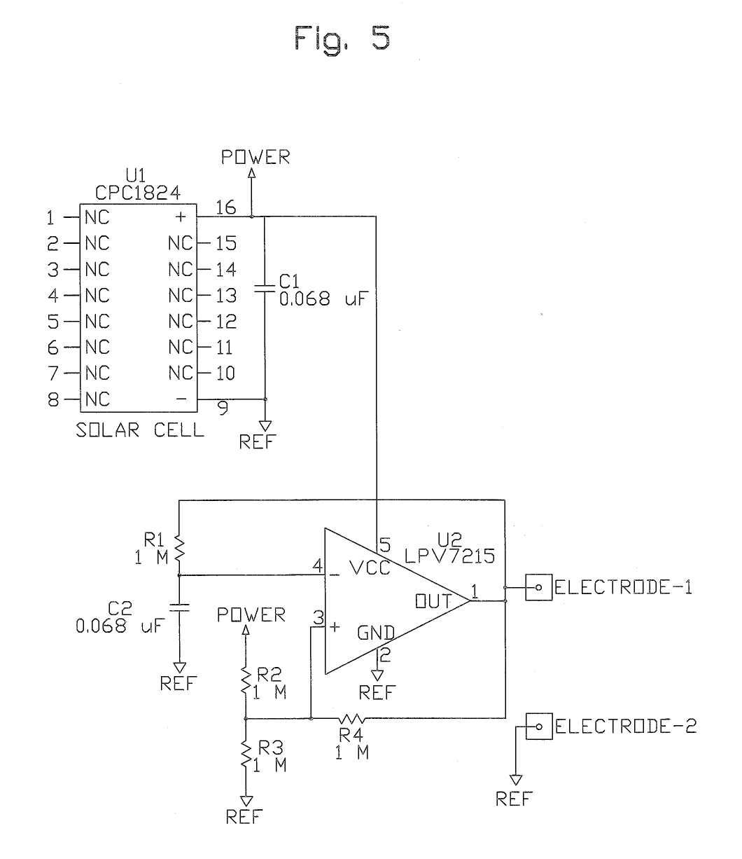

FIG. 5 shows a schematic of the circuit.

The embodiment shown here uses a Clare CPC1824 photovoltaic array and a Texas Instruments LPV7215 comparator configured in a standard oscillator circuit. Other embodiments could use other forms of photovoltaic array. While the embodiment shown here uses an oscillator as a pulse generator, other forms of pulse-generating circuit could be used, such as a logic circuit or microcomputer.

The embodiment shown here is a flat, circular device with a diameter of less than 15 mm and a thickness of less than 5 mm. This makes it well suited as a wearable device that can remain in place for long periods while the user goes about their normal activities.

I Claim:

1. A wearable apparatus for providing electrical stimulation to the skin, comprising:
a photovoltaic array that provides a source of electrical power
an electrical circuit that generates a pulsed output
at least two electrodes for applying the pulsed output to the skin

2. The apparatus of claim 1, wherein the pulsed output is between 1 Hz and 300 Hz.

3. The apparatus of claim 2, wherein the pulsed output is in the form of a square wave.
